(19) United States
(12) Patent Application Publication
(10) Pub. No.: US 2006/0263308 A1
(43) Pub. Date: Nov. 23, 2006

(54) Method for improving skin radiance and luminosity

(76) Inventors: Ivonne Brown, Roosevelt, NY (US); Ida Marie Sandewicz, Monroe Township, NJ (US); Tatyana Zamyatin, Princeton Junction, NJ (US); Josephine Obuobisah, East Orange, NJ (US)

Correspondence Address: Julie Blackburn;Revlon Consumer Products Corporation, Law Department, 237 Park Avenue, New York, NY, 10017, US

(21) Appl. No.: 11/177,172

(22) Filed: Jul. 8, 2005

Related U.S. Application Data
(60) Provisional application No. 60/681,619, filed on May 17, 2005.

(51) Int. Cl. A61K 8/19; A61K 8/35

(52) U.S. Cl. 424/59; 424/63; 424/64; 424/70.7

(57) ABSTRACT

A method for improving skin radiance and luminosity comprising treating with skin with a cosmetic composition comprising a self-tanning ingredient that is present in an amount insufficient to provide a visually perceptible tan on the skin.

This application claims priority from U.S. provisional patent application Ser. No. 60/681,619, filed May 17, 2005

### TECHNICAL FIELD

The invention is in the field of color cosmetic compositions, specifically those used to improve skin radiance, luminosity, and appearance.

### BACKGROUND OF THE INVENTION

Cosmetics manufacturers are always looking for ways to improve the conditioning, moisturizing, and hydrating properties of color cosmetics. Consumers want color cosmetics that will provide color to the skin as well as provide skin benefits similar to those found with prestige skin creams and lotions. For example, foundation makeup is an excellent vehicle for providing color to the skin and at the same time providing skin care benefits such as firming, toning, wrinkle reduction, and so on. Cosmetics companies are interested in making foundation makeup and other color cosmetics applied to the face as multi-purpose as possible so that the consumer can get the maximum benefit from one product.

One skin characteristic that is associated with youthfulness is skin radiance and luminosity. The term “radiance” means that the skin glows. The term “luminous” means that the skin emits a soft, subdued light. Both radiance and luminosity are often seen in childrens' skin. These characteristics are often lost as we age and skin becomes thicker and keratinous cells do not slough as readily from skin surfaces. There is always interest in locating raw materials for use in skin care products and color cosmetics that will enhance radiance and luminosity of skin.

Self-tanning compositions are well known in the art, and typically contain an ingredient that reacts with the proteins found in skin to turn skin a subtle brown color that approximates a tan. This reaction is typically referred to as a “Maillard reaction”. The tanning ingredient most commonly used in self-tanners is dihydroxyacetone, also referred to as DHA. DHA induced tans last only a few days, or as long as it takes for the superficial keratinous cells that have reacted with the DHA to slough off.

It has most unexpectedly been found that skin luminosity and radiance can be improved by treating skin with color cosmetics such as foundation makeup, foundation primer, or skin creams and lotions containing a self-tanning ingredient in very small amounts—in particular in amounts insufficient to provide a visually perceptible tan to the skin.

Accordingly, it is an object of the invention to provide a method for improving skin radiance and luminosity by treating the skin with a cosmetic composition containing a self-tanning ingredient in an amount insufficient to provide a visually perceptible tan to the skin.

### SUMMARY OF THE INVENTION

The invention is directed to a method for improving skin radiance and luminosity comprising treating skin with a cosmetic composition comprising a self-tanning ingredient that is present in an amount insufficient to provide a visually perceptible tan on the skin.

The invention is also directed to a color cosmetic composition containing a self-tanning agent in combination with a silicone surfactant having a siloxane backbone having attached thereto at least one polyether group and at least one fatty alkyl group.

### DETAILED DESCRIPTION

All percentages mentioned herein are percentages by weight unless otherwise indicated.

The term “visually perceptible” means that the skin treated with the composition containing the self-tanning agent will appear to be tanned, or darker in color, when visually observed with the naked eye.

In general, the time period in which no visually perceptible tan will be observed may range from the time immediately after application up to about 10 days, preferably about 5 days, more preferably about 3 days, more preferably about 1 day or 24 hours, when the composition is applied once per day.

The term “radiance” means that the skin exhibits a subtle overall glow.

The term “luminosity” means that the skin exhibits a soft, subdued light.

The invention is directed to a method for improving the radiance and luminosity of skin by treating the skin with a cosmetic composition containing a self-tanning ingredient in an amount insufficient to provide a visually perceptible tan on the skin. Yet, the amount of the self-tanning ingredient must be present in the composition in an amount sufficient to improve skin luminosity and radiance.

The color cosmetic composition may be in the anhydrous or emulsion form.

I. Self-Tanning Ingredient

Generally, suitable self-tanning ingredients include those that react with the proteins and amino acids on the skin surface to brown the skin in what is referred to as the Maillard reaction. Ingredients that are known to react with skin proteins in this manner include dihydroxyacetone of DHA. The amount of self-tanning ingredient that is present in the composition must be insufficient to cause the skin to exhibit a visually perceptible tanning or browning when treated with the composition containing the agent, but yet sufficient to improve the radiance and luminosity of the skin. The amount of self-tanning agent present in the composition that will provide this type of result may range from about 0.001-3%, preferably from about 0.005-2.5%, more preferably from about 0.01-1.5% by weight of the total composition. The amount of self-tanning agent that is present and provides the desired end result also may depend on the other ingredients in the formulation. Preferably, the composition of the invention will not provide a visually perceptible tan on the skin when used under circumstances normal for the use of foundation makeup. More preferably, the composition will not provide a visually perceptible tan on the skin when used once per day for a period of from about one to thirty days or more.

II. Cosmetic Compositions

The self-tanning agent may be incorporated into a wide variety of color cosmetic compositions in either the anhydrous, gel, or emulsion form. If in emulsion form, the emulsions may be water-in-oil or oil-in-water.

A. Emulsion Color Cosmetics

Suitable color cosmetics in the emulsion form include foundation makeup compositions, primers, concealers, blushes, eyeshadows, lipcolor, lipliner, and the like. Such emulsion compositions contain water, oil, pigments, and other ingredients.

1. Water and Water Phase Ingredients

The emulsion color cosmetic compositions that may be used in the method of the invention comprise 1-95%, preferably 5-80%, more preferably 10-70% by weight of the total composition of water. The water forms a phase which may contain other ingredients that are soluble in the aqueous phase such as mono-, di-, or polyhydric alcohols, water soluble plant extracts, and the like.

(a). Alcohols

Suitable alcohols include mono-, di-, or polyhydric alcohols such as ethanol, propanol, butylene glycol, propylene glycol, benzyl alcohol, butyl alcohol, hexanol, and the like. If one or more of these alcohols are present in the composition, a range of 0.01-10%, preferably, 0.05-8%, more preferably 0.1-5% by weight of the total composition is suggested. Particularly preferred alcohols are propylene glycol, butylene glycol, or mixtures thereof.

(b). Plant Extracts

A variety of water soluble plant extracts may be solubilized in the aqueous phase of the compositions used in the method of the invention. Examples of such extracts include extracts of flowers or plants such as chamomile, aloe, apple, lady's slipper, meadowsweet, lime, lemon, lilac, oat bran, mulberry root, grapefruit, saxifraga sarmentosa, tannic acid, kojic acid, and so on. Suitable ranges of water soluble plant extracts are 0.001-8%, preferably 0.005-5%, more preferably 0.01-4% by weight of the total composition. Preferred extracts are chamomile, lemon, mulberry root, grapefruit, saxifraga sarmentosa, tannic acid, kojic acid, and mixtures thereof. Particularly preferred are chamomile extract, and an extract sold by Coletica/Bioetica under the tradename Phytoclar, which is a mixture of water, mulberry root extract, butylene glycol, lemon extract, grapefruit extract, saxifraga sarmentose extract, tannic acid, kojic acid, and xanthan gum.

(c). Emulsion Stabilizers

It also may be desirable to include one or more emulsion stabilizers in the aqueous phase of the compositions used in the method of the invention. Suitable emulsion stabilizers are salts such as potassium chloride, sodium chloride, magnesium sulfate, ammonium chloride, sodium chloride, and the like. If present in the composition, a suitable range for the emulsion stabilizer comprises 0.001-5%, preferably 0.005-4%, more preferably 0.1-3% by weight of the total composition. The preferred emulsion stabilizer is sodium chloride.

2. Oil Phase

The emulsion color cosmetic compositions used in the method of the invention may comprise 0.1-50%, preferably 1-30%, more preferably 5-25% by weight of the total composition of an oil phase which comprises silicone oil having dispersed therein one or more colorants. The oil phase may additionally contain one or more organic oils, provided that the silicone oil and organic oils are compatible, i.e. soluble in each other.

(a). Silicone Oil

The silicone oil may be volatile, non-volatile or a mixture of both, provided that the silicone oils are soluble in each other, and in the oil phase of the composition. Suitable volatile silicones include cyclic silicones (or cyclomethicones) are of the general formula: 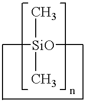
where n 3-7.

Linear volatile silicones in accordance with the invention have the general formula:
(CH₃)₃Si—O—[Si(CH₃)₂—O]ₙ—Si(CH₃)₃
where n=0-6, preferably 0-5.

Linear and cyclic volatile silicones are available from various commercial sources including Dow Corning Corporation and General Electric. The Dow Corning volatile silicones are sold under the tradenames Dow Corning 244, 245, 344, and 200 fluids. These fluids comprise hexamethyldisiloxane, octamethyltrisiloxane, decamethyltetrasiloxane, dodecamethylpentasiloxane, octamethylcyclotetrasiloxane, decamethylcyclopentasiloxane, dodecamethylhexasiloxane, and mixtures thereof.

Suitable nonvolatile silicones include water insoluble silicones having a viscosity ranging from about 10 to 600,000 centistokes, preferably 20 to 100,000 centistokes at 25° C. Suitable water insoluble silicones include cetyl dimethicone, dimethicone, phenyl trimethicone, phenyldimethicone, diphenyl dimethicone, and mixtures thereof.

Also suitable as the nonvolatile silicone oil are various fluorinated silicones such as trimethylsilyl endcapped fluorosilicone oil, polytrifluoropropylmethylsiloxanes, and similar silicones such as those disclosed in U.S. Pat. No. 5,118,496 which is hereby incorporated by reference.

Preferably, the color cosmetic compositions used in the method of the invention comprise a mixture of volatile and non-volatile silicones, in particular, about 0.5-20% by weight of the total composition of volatile silicone oil, and about 0.5-15% nonvolatile silicone. The presence of the volatile silicone enables the makeup to dry on the skin in an appropriate period of time, and minimizes the heavy, greasy feel that is occasionally found with nonvolatile oils. The remaining nonvolatile oil phase acts to plasticize the film formed on the skin by the dried aqueous phase containing the solubilized soy protein.

3. Colorants

Dispersed in the oil or aqueous phase of the composition are one or more colorants. Suitable colorants may be inorganic or organic pigments and powders. The organic pigments are generally various aromatic types including azo, indigoid, triphenylmethane, anthraquinone, and xanthine dyes which are designated as D&C and FD&C blues, browns, greens, oranges, reds, yellows, etc. Organic pigments also generally consist of insoluble metallic salts of certified color additives, referred to as the Lakes. Inorganic pigments include iron oxides, ultramarines, chromium, chromium hydroxide colors, and mixtures thereof. Preferably, the pigments may be coated with one or more ingredients that cause the pigments to be hydrophobic. Suitable coating materials that will render the pigments more lipophilic in nature include silicones, lecithin, amino acids, phospholipids, inorganic and organic oils, polyethylene, and other polymeric materials. Particularly preferred are silicone treated pigments as disclosed in U.S. Pat. No. 5,143,722, which is hereby incorporated by reference.

4. Other Ingredients

The oil phase of the compositions used in the method of the invention may contain additional ingredients that are soluble or dispersible in the oil phase, such as waxes, oil soluble synthetic polymers, particulates, and so on.

(a). Particulates

The oil phase preferably contains one or more particulates (including pigments or powders) that serve as powders, colorants, fillers, or sunscreens in the composition. These powders or fillers are present for providing or adjusting the color of the composition, and in some cases may provide a sunscreen effect by physical blocking of UV radiation. Preferably, the particle size of the particulates ranges from 0.05 to 100 microns, and are present in ranges of 0.1-20%, preferably 0.5-15%, more preferably 1-10% by weight of the total composition. Examples of particulates include white or non-pigmentitious powders include bismuth oxychloride, titanated mica, fumed silica, spherical silica, polymethylmethacrylate, micronized teflon, boron nitride, acrylate copolymers, aluminum silicate, aluminum starch octenylsuccinate, bentonite, calcium silicate, cellulose, chalk, corn starch, diatomaceous earth, fuller's earth, glyceryl starch, hectorite, hydrated silica, kaolin, magnesium aluminum silicate, magnesium trisilicate, maltodextrin, montmorillonite, microcrystalline cellulose, rice starch, silica, talc, mica, titanium dioxide, zinc laurate, zinc myristate, zinc rosinate, alumina, attapulgite, calcium carbonate, calcium silicate, dextran, kaolin, nylon, silica silylate, silk powder, sericite, soy flour, tin oxide, titanium hydroxide, trimagnesium phosphate, walnut shell powder, or mixtures thereof. While titanium dioxide is commonly considered to be a white pigment when used in paints, in color cosmetic compositions it is used more for its ability to mute color, and/or provide an opaque or semi-opaque finish, or provide sunscreen protection, then as a colorizing ingredient. The above mentioned particulates may be surface treated with lecithin, amino acids, mineral oil, silicone oil or various other agents either alone or in combination, which coat the powder surface and render the particles more lipophilic in nature.

It is particularly preferred that the compositions used in the method of the invention comprise very fine particle zinc oxide and/or titanium dioxide, in addition to the other pigments and particulates which may be present. The mixture of zinc oxide and titanium dioxide causes the makeup composition to exhibit a very high sun protective factor (SPF), as high as 15 to 20 SPF. Preferred particle sizes of the zinc oxide and titanium dioxide are 0.005 to 10 microns. Preferably the compositions of the invention contain 1-15% by weight of the composition of zinc oxide, titanium dioxide, or mixtures thereof, having a particle size of 0.005 to 10 microns and providing makeup having an SPF of 15 to 20, preferably 20.

A variety of organic or inorganic pigments are suitable, including iron oxides, FD&C colors, or D&C colors such as red, yellow, black, iron oxides and mixtures thereof.

(b). Organic Oils

The oil phase may additionally comprise one or more organic oils. Examples of suitable organic oils include hydrocarbons, esters, and the like. Suitable hydrocarons include paraffinic hydrocarbons having 9 to 70 carbon atoms, C₂₀₋₄₅ olefins, isododecane, isobutene, isoeicosane, isohexadecane, mineral oil, squalene, squalane, and the like.

Also suitable are various esters that are liquid at room temperature. Suitable esters include guerbet esters, which are generally defined as esters which are formed by the reaction of a guerbet alcohol (which is a branched chain alcohol) having the general formula: 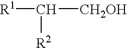
with a carboxylic acid having the general formula: 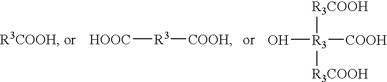
wherein R¹ and R² are each independently a C₄₋₂₀ alkyl and each R³ is a substituted or unsubstituted C₁₋₅₀ straight or branched chain alkyl or alkylene group, or phenyl, wherein the substituents are halogen, hydroxyl, carboxyl, or alkylcarbonyloxy.

Other suitable esters include those having the formula RCO—OR′ wherein R and R′ are each independently a C₁₋₂₅, preferably a C₄₋₂₀, straight or branched chain alkyl, alkenyl or alkoxycarbonylalkyl or alkylcarbonyloxyalkyl. Preferred are esters that are the reaction product of of a branched chain fatty acid and a branched or straight chain fatty alcohol, preferably a branched chain fatty alcohol. Examples of such esters include isotridecyl isononanoate, isostearyl neopentanoate, tridecyl neopentanoate, cetyl octanoate, cetyl ricinoleate, decyl isostearate, isodecyl oleate, isodecyl neopentanoate, isohexyl neopentanoate, tridecyl octanoate, and so on.

Other suitable esters include naturally occuring glyceryl esters of fatty acids, or triglycerides. Both vegetable and animal sources may be used. Examples of such oils include castor oil, lanolin oil, triisocetyl citrate, C₁₀₋₁₈ triglycerides, caprylic/capric/triglycerides, coconut oil, corn oil, cottonseed oil, linseed oil, mink oil, olive oil, palm oil, illipe butter, rapeseed oil, soybean oil, sunflower seed oil, walnut oil, and the like.

Also suitable are synthetic or semi-synthetic glyceryl esters, e.g. fatty acid mono-, di-, and triglycerides which are natural fats or oils that have been modified, for example, acetylated castor oil, glyceryl stearate, glyceryl dioleate, glyceryl distearate, glyceryl trioctanoate, glyceryl linoleate, glyceryl myristate, glyceryl isostearate, PEG castor oils, PEG glyceryl oleates, PEG glyceryl stearates, PEG glyceryl tallowates, and so on.

(c). Waxes

The compositions used in the method of the invention may also contain one or more waxes which are solid or semi-solid at room temperature, provided such waxes and soluble or miscible with the oil phase when the wax and the oil phase are heated together to a temperature sufficient to melt the wax. Suggested ranges of wax are 0.1-20%, preferably 0.5-15%, more preferably 1-10% by weight of the total composition. The waxes preferably have a melting point of about 39 to 135° C., preferably in the range of 45 to 95° C., most preferably 55 to 95° C. Suitable waxes generally include animal waxes, plant waxes, mineral waxes, silicone waxes, synthetic waxes, and petroleum waxes. More specifically, these waxes include tribehenin, bayberry, beeswax, candelilla, carnauba, ceresin, cetyl esters, hydrogenated jojoba oil, hydrogenated jojoba wax, hydrogenated microcrystalline wax, hydrogenated rice bran wax, japan wax, jojoba butter, jojoba esters, jojoba wax, lanolin wax, microcrystalline wax, mink wax, montan acid wax, montan wax, ouricury wax, ozokerite, paraffin, cetyl alcohol, beeswax, PEG-20 sorbitan beeswax, PEG-8 beeswax, rice bran wax, shellac wax, spent grain wax, sulfurized jojoba oil, synthetic beeswax, synthetic candelilla wax, synthetic carnauba wax, synthetic japan wax, synthetic jojoba oil, synthetic wax, polyethylene, stearoxy dimethicone, dimethicone behenate, stearyl dimethicone, and the like, as well synthetic homo- and copolymer waxes such as PVP/eicosene copolymer, PVP/hexadecene copolymer, and the like. Particularly preferred is where the wax is an organic wax, tribehenin.

4. Surfactant

The emulsion compositions used in the method of the invention may comprise an effective amount of a surfactant which is capable of causing the water phase and the oil phase to form an emulsion having stability for two weeks at 50° C. Suggested ranges of surfactant are in the range of about 0.1-20%, preferably 0.5-15%, more preferably 1-10% by weight of the total composition of one or more surfactants. Suitable surfactants include organic or silicone surfactants, which may be anionic, cationic, nonionic, zwitterionic, or amphoteric. Preferably the surfactants are nonionic organic or silicone surfactants.

Examples of nonionic organic surfactants include alkoxylated alcohols, or ethers, formed by the reaction of an alcohol with an alkylene oxide, usually ethylene or propylene oxide. Preferably the alcohol is either a fatty alcohol having 6 to 30 carbon atoms. Examples of such ingredients include Beheneth 5-30, which is formed by the reaction of behenyl alcohol and ethylene oxide where the number of repeated ethylene oxide units is 5 to 30; Ceteareth 2-100, formed by the reaction of a mixture of cetyl and stearyl alcohol with ethylene oxide, where the number of repeating ethylene oxide units in the molecule is 2 to 100; Ceteth 1-45 which is formed by the reaction of cetyl alcohol and ethylene oxide, and the number of repeating ethylene oxide units is 1 to 45, laureth, 1-100 where the number of repeating ethylene oxide units is 1 to 100, and so on. Other alkoxylated alcohols are formed by the reaction of fatty acids and mono-, di- or polyhydric alcohols with an alkylene oxide. For example, the reaction products of C₆₋₃₀ fatty carboxylic acids and polyhydric alcohols which are monosaccharides such as glucose, galactose, methyl glucose, and the like, with an alkoxylated alcohol.

Also suitable as the nonionic surfactant are alkyoxylated carboxylic acids, which are formed by the reaction of a carboxylic acid with an alkylene oxide or with a polymeric ether. The resulting products have the general formula: 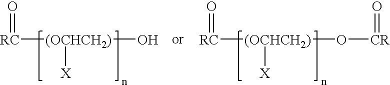
where RCO is the carboxylic ester radical, X is hydrogen or lower alkyl, and n is the number of polymerized alkoxy groups. In the case of the diesters, the two RCO— groups do not need to be identical. Preferably, R is a C₆₋₃₀ straight or branched chain, saturated or unsaturated alkyl, and n is from 1-100.

Also suitable as the nonionic surfactant are monomeric, homopolymeric and block copolymeric ethers. Such ethers are formed by the polymerization of monomeric alkylene oxides, generally ethylene or propylene oxide. Such polymeric ethers have the following general formula: 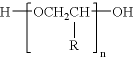
wherein R is H or lower alkyl and n is the number of repeating monomer units, and ranges from 1 to 500.

Other suitable nonionic surfactants include alkoxylated sorbitan and alkoxylated sorbitan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.

Also suitable as nonionic surfactants are silicone surfactants, which are defined as silicone polymers, which have at least one hydrophilic radical and at least one lipophilic radical. The silicone surfactant that may be used in the compositions are organosiloxane polymers that may be a liquid or solid at room temperature. The organosiloxane surfactant is generally a water-in-oil or oil-in-water type surfactant which is, and has an Hydrophile/Lipophile Balance (HLB) of 2 to 18. Preferably the organosiloxane is a nonionic surfactant having an HLB of 2 to 12, preferably 2 to 10, most preferably 4 to 6. The HLB of a nonionic surfactant is the balance between the hydrophilic and lipophilic portions of the surfactant and is calculated according to the following formula:
HLB=7+11.7×log M_{w}/Mₒ
where M_{w} is the molecular weight of the hydrophilic group portion and Mₒ is the molecular weight of the lipophilic group portion.

The polymeric organosiloxane surfactant used in the invention may have any of the following general formulas:
MₓQ_{y}, or
MₓT_{y}, or
MDₓD′_{y}″_{z}M
wherein each M is independently a substituted or unsubstituted trimethylsiloxy endcap unit. If substituted, one or more of the hydrogens on the endcap methyl groups are substituted, or one or more methyl groups are substituted with a substituent that is a lipophilic radical, a hydrophilic radical, or mixtures thereof. T is a trifunctional siloxy unit having the empirical formula RR′SiO_{1.5} or RRSiO_{1.5}. Q is a quadrifunctional siloxy unit having the empirical formula SiO₂, and D, D′, D″, x, y, and z are as set forth below, with the proviso that the compound contains at least one hydrophilic radical and at least one lipophilic radical. Preferred is a linear silicone of the formula:
MDₓD′_{y}D″_{z}M
wherein M=RRRSiO_{1/2}
D and D′=RR′SiO_{2/2}
D″=RRSiO_{2/2}
x, y, and z are each independently 0-1000,
where R is methyl or hydrogen, and R′ is a hydrophilic radical or a lipophilic radical, with the proviso that the compound contains at least one hydrophilic radical and at least one lipophilic radical.
   Most preferred is wherein
M=trimethylsiloxy
D=Si[(CH₃)][(CH₂)ₙCH₃]O_{2/2} where n=0-40,
D′=Si [(CH₃)][(CH₂)ₒ—O-PE)]O_{2/2} where PE is (—C₂H₄O)ₐ(—C₃H₆O)_{b}H, o=0-40,
a=1-100 and b=1-100, and
D″=Si (CH₃)₂O_{2/2}

More specifically, suitable silicone surfactants have the formula: 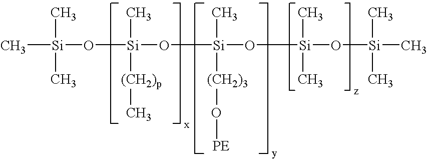
wherein p is 0-40, and
PE is (—C₂H₄O)ₐ(—C₃H₆O)_{b}—H
where x, y, z, a, and b are such that the maximum molecular weight of the polymer is approximately 50,000.

Another type of preferred organosiloxane emulsifier suitable for use in the compositions of the invention are emulsifiers sold by Union Carbide under the Silwet™ trademark, which are referred to by the CTFA term “dimethicone copolyol”.

Also suitable as nonionic silicone surfactants are hydroxy-substituted silicones such as dimethiconol, which is defined as a dimethyl silicone substituted with terminal hydroxy groups.

Most preferred are polyether or polyglycerin modified silicones that may be linear, branched, or crosspolymeric types. Examples of polyether modified silicones include PEG-11 methyl ether dimethicone, PEG/PPG-20/22 butyl ether dimethicone, PEG-9 dimethicone, PEG-3 dimethicone, PEG-9 methyl ether dimethicone, PEG-10 dimethicone. Examples of suitable alkyl/polyether modified silicones are PEG/PPG-10/3 oleyl ether dimethicone. Examples of branched chain polyether modified silicones include PEG-9 polydimethylsiloxyethyl dimethicone, or lauryl PEG-9 polydimethylsiloxy dimethicone. Also suitable are various crosspolymeric types such as dimethicone and dimethicone/PEG-10/15 crosspolymer, PEG-15/lauryl dimethicone crosspolymer, PEG-15 lauryl dimethicone crosspolymer, or mixtures thereof. Also suitable are polyglycerin-modified silicone surfactants such as polyglyceryl-3 disiloxane dimethicone, polyglyceryl-3 polydimethylsiloxyethyl dimethicone; or alkyl modified types such as lauryl polyglyceryl-3 polydimethylsiloxyethyl dimethicone. Crosspolymeric glycerin substituted silicones are also suitable, such as dimethicone/polyglycerin-3 crosspolymer, lauryl dimethicone/polyglycerin-3 crosspolymer, and so on. Such silicones may be generally described as those having a linear or crosslinked siloxane backbone having substituent groups selected from polyether, polyglycerin, alkyl, or combinations thereof. The term “polyether” means repeating alkylene glycol groups such as ethylene or propylene glycol, more specifically PEG with from about 1 to 50 repeating ethylene glycol units. The term “polyglycerin” means repeating glycerin units. The term “alkyl” means C₁₋₅₀ straight or branched chain alkyl groups with C₆₋₂₂ fatty alkyl groups being particularly preferred. These types of substituted silicone surfactants are sold by Shin-Etsu silicones under the tradenames KF-6011, KF-6012, KF-6013, KF-6015, KF-6016, KF-6017, KF-6026, KF-6028, KF-6100, KF-6104, KF-6038, KF-6105, KSG-210, KSG-710, KSG-310, KSG-320, KSG-330, KSG-340, KSG-810, KSG-820, KSG-830, KSG-840. Another type of such surfactant is sold by Active Concepts under the trade name Silderm Emulsifying CS.

Other examples of silicone surfactants are those sold by Dow Corning under the tradename Dow Corning 3225C or 5225C Formulation Aid, Dow Corning 190 Surfactant, Dow Corning 193 Surfactant, Dow Corning Q2-5200, and the like are also suitable. In addition, surfactants sold under the tradename Silwet by Union Carbide, and surfactants sold by Troy Corporation under the Troysol tradename, those sold by Taiwan Surfactant Co. under the tradename Ablusoft, those sold by Hoechst under the tradename Arkophob, are also suitable for use in the invention.

5. Other Ingredients

The compositions of the invention may contain other ingredients such as preservatives, antioxidants, vitamins, and so on.

(a). Preservatives

The composition used in the method of the invention may contain 0.0001-8%, preferably 0.001-6%, more preferably 0.005-5% by weight of the total composition of preservatives. A variety of preservatives are suitable, including such as benzoic acid, benzyl alcohol, benzylhemiformal, benzylparaben, 5-bromo-5-nitro-1,3-dioxane, 2-bromo-2-nitropropane-1,3-diol, butyl paraben, calcium benzoate, calcium propionate, captan, chlorhexidine diacetate, chlorhexidine digluconate, chlorhexidine dihydrochloride, chloroacetamide, chlorobutanol, p-chloro-m-cresol, chlorophene, chlorothymol, chloroxylenol, propyl paraben, methyl paraben, benzoic acid, m-cresol, o-cresol, DEDM Hydantoin, DEDM Hydantoin dilaurate, dehydroacetic acid, diazolidinyl urea, dibromopropamidine diisethionate, DMDM Hydantoin, and all of those disclosed on pages 570 to 571 of the CTFA Cosmetic Ingredient Handbook, Second Edition, 1992, which is hereby incorporated by reference.

(b). Vitamins and Antioxidants

The composition used in the method of the invention may contain vitamins and/or coenzymes, as well as antioxidants. If so, 0.001-10%, preferably 0.01-8%, more preferably 0.05-5% by weight of the total composition are suggested. Suitable vitamins include the B vitamins such as thiamine, riboflavin, pyridoxin, and so on, as well as coenzymes such as thiamine pyrophoshate, flavin adenin dinucleotide, folic acid, pyridoxal phosphate, tetrahydrofolic acid, and so on. Also Vitamin A and derivatives thereof are suitable. Examples are Vitamin A palmitate, acetate, or other esters thereof, as well as Vitamin A in the form of beta carotene. Also suitable is Vitamin E and derivatives thereof such as Vitamin E acetate, nicotinate, or other esters thereof. In addition, Vitamins D, C, and K, as well as derivatives thereof are suitable. Particularly preferred are derivatives of vitamins C, E, and A such as magnesium ascorbyl phosphate, retinyl palmitate, tocopheryl acetate, and mixtures thereof.

Suitable antioxidants are ingredients that assist in preventing or retarding spoilage. Examples of antioxidants suitable for use in the compositions of the invention are potassium sulfite, sodium bisulfite, sodium erythrobate, sodium metabisulfite, sodium sulfite, propyl gallate, cysteine hydrochloride, butylated hydroxytoluene, butylated hydroxyanisole, and so on.

(c). PH Adjusters

Also suitable are ingredients that will adjust the pH and assist in maintaining pH within certain ranges. Suitable pH adjusters include carboxylic acids such as lactic acid and the like. In the most preferred embodiment of the composition the pH adjusters maintain the pH of the composition at less than 4, which provides the optimum stability for DHA.

B. Anhydrous Color Cosmetics

Various types of anhydrous color cosmetics are also suitable for use in the method of the invention. Typically such anhydrous compositions include lipsticks, blushes, eyeshadows, and the like.

Typically, anhydrous color cosmetics contain an oily phase in combination with particulates. Suitable oils and particulates are as discussed above with respect to the emulsion color cosmetics, and in the same general percentage ranges.

The invention will be further described in connection with the following examples, which are set forth for the purposes of illustration only.

### EXAMPLE 1

A foundation makeup composition suitable for use with the method of the invention was prepared as follows:

| | | w/w % |
|---|---|---|
| 1 | Cyclomethicone | QS |
| 1 | Lauryl PEG-9 polydimethylsiloxyethyl dimethicone | 5.00 |
| 1 | Titanium dioxide, alumina, methicone | 5.00 |
| 1 | Titnaium dioxide, methicone | 3.00 |
| 1 | Iron oxides, methicone | 1.54 |
| 1 | Silica silylate | 0.05 |
| 1 | Mica, methicone | 0.01 |
| 1 | Talc, methicone | 3.50 |
| 2 | Sorbitan trioleate | 0.30 |
| 2 | 33% propyl paraben in laureth-7 | 0.75 |
| 2 | Tribehenin | 0.30 |
| 2 | Dimethicone | 3.00 |
| 2 | PPG-3 myristyl ether neoheptanoate | 2.00 |
| 2 | Cyclomethicone/polymethylsilsesquioxane | 1.50 |
| 3 | Water | 33.82 |
| 3 | Methyl paraben | 0.12 |
| 3 | Butylene glycol | 7.00 |
| 3 | Sodium chloride | 1.00 |
| 3 | Tetrasodium EDTA | 0.01 |
| 3 | Magnesium sulfate | 1.00 |
| 3 | Lactic acid | 0.05 |
| 3 | Dihydroxacetone | 1.00 |
| 4 | Acrylonitrile/methacrylonitrile/methyl methacrylate | 0.4 |
| | copolymer, iron oxides, talc, water | |
| 4 | Dimethicone | 4.00 |
| 4 | Ethylene brassylate | 0.05 |

While the invention has been described in connection with the preferred embodiment, it is not intended to limit the scope of the invention to the particular form set forth but, on the contrary, it is intended to cover such alternatives, modifications, and equivalents as may be included within the spirit and scope of the invention as defined by the appended claims.

We claim:

1. A method for improving skin radiance and luminosity comprising treating with skin with a color cosmetic composition comprising a self-tanning ingredient that is present in an amount insufficient to provide a visually perceptible tan on the skin.

2. The method of claim 1 wherein the self-tanning ingredient is dihydroxyacetone.

3. The method of claim 1 wherein the color cosmetic composition is a color-cosmetic composition containing iron oxide pigments.

4. The method of claim 3 wherein the color cosmetic composition is anhydrous.

5. The method of claim 3 wherein the color cosmetic composition is an emulsion.

6. The method of claim 1 wherein the color cosmetic composition is a blush, eyeshadow, foundation makeup, eyeliner, lipliner, mascara, lipstick, or lip gloss.

7. The method of claim 5 wherein the color cosmetic composition is a water and oil emulsion foundation makeup composition.

8. The method of claim 7 wherein the foundation makeup is a water in oil emulsion.

9. The method of claim 1 wherein the self tanning ingredient tans skin by reacting with the amino acids present in skin keratin.

10. The method of claim 1 wherein the amount of self tanning ingredient ranges from about 0.0001 to 3% by weight of the total composition.

11. The method of claim 1 wherein the color cosmetic composition is a water in silicone oil emulsion comprising from about 0.1-85% water, 0.1-75% silicone oil, and from about 0.01-45% pigments, all percentages by weight of the total composition.

12. The method of claim 1 wherein the skin radiance is improved.

13. The method of claim 1 wherein skin luminosity is improved.

14. The method of claim 1 wherein the cosmetic composition is a water in silicone oil emulsion foundation makeup composition comprising, by weight of the total composition, from about 0.01-99% water, from about 0.01-85% silicone oil, and from about 0.001-20% of a polyorganosiloxane-polyoxyalkylene surfactant; and from about 0.01-50% particulate matter.

15. The method of claim 14 wherein the particulate matter comprises a mixture of pigments and powders.

16. The method of claim 14 wherein the polyorganosiloxane-polyoxyalkylene surfactant comprises dimethicone copolyol, cetyl dimethicone copolyol, or mixtures thereof.

17. The method of claim 14 wherein the composition further comprises one or more humectants.

18. The method of claim 17 wherein the humectants are present ranging from about 0.01-20% by weight of the total composition.

19. The method of claim 1 wherein the cosmetic composition is a skin cream or lotion containing from about 0.01-99% water, 0.01-85% silicone oil, from about 0.01-20% nonionic surfactant, and from about 0.01-20% of one or more skin benefit agents.

20. The method of claim 19 wherein the skin benefit agents are
botanicals.
